(19) United States
(12) Patent Application Publication
(10) Pub. No.: US 2026/0248880 A1
(43) Pub. Date: Aug. 27, 2026

(54) NANOPARTICLE COMPOSITIONS AND USES THEREOF FOR REACTIVATION OF LATENT HIV

(71) Applicants: The Wistar Institute of Anatomy and Biology, Philadelphia, PA (US); The Trustees of the University of Pennsylvania, Philadelphia, PA (US)

(72) Inventors: Luis Montaner, Philadelphia, PA (US); Daniel Claiborne, Philadelphia, PA (US); Hamideh Parhiz, Gladwyne, PA (US); Tyler E. Papp, Philadelphia, PA (US)

(21) Appl. No.: 19/159,195

(22) PCT Filed: Feb. 26, 2024
(86) PCT No.: PCT/US2024/017238

§ 371 Date: Aug. 22, 2025

Related U.S. Application Data
(60) Provisional application No. 63/487,006, filed on Feb. 26, 2023.

(51) Int. Cl. A61K 38/16; A61K 9/51; A61P 31/18

(52) U.S. Cl. CPC A61K 38/162; A61K 9/5123; A61P 31/18

(57) ABSTRACT

Provided herein are nanoparticles comprising a messenger RNA (mRNA) encoding an HIV tat protein and methods of administering the nanoparticles to a patient for treating HIV.

### STATEMENT REGARDING FEDERALLY SPONSORED RESEARCH OR DEVELOPMENT

This invention was made with government support under AI164570 awarded by the National Institutes of Health. The government has certain rights in the invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “WST-195.PCT.xml”, was created Feb. 26, 2024 and is 3,196 bytes.

### BACKGROUND OF THE INVENTION

HIV infects 36.9 million people worldwide, 2.6 million of whom are children. Combination antiretroviral therapy (ART) has dramatically reduced morbidity and mortality for HIV-infected individuals in resource-rich countries with access to healthcare. However, more than 22 million people do not have access to ART, including 1.8 million children (UNAIDS report, 2015). Moreover, ART requires lifelong administration of at least three different medicines and does not eradicate HIV, which continues to cause immune activation, inflammation, and ongoing damage to multiple organs systems. Critically, ART does not fully prevent pathology or restore a normal lifespan in HIV-infected patients. These limitations are due to drug side effects and/or incomplete viral suppression, particularly in viral anatomical sanctuaries. Moreover, with continuous and expanded usage of ART, HIV is becoming progressively more drug resistant. This erodes the efficacy of ART, especially in settings with limited resources or inadequate access to therapy. Thus, lifelong ART is not a sustainable solution to treating HIV/AIDS at either an individual or global scale, and curative therapies are urgently needed.

The main barrier to HIV eradication is the ability of HIV to establish persistent infection in long-lived CD4+ T cells, which persist in the blood and at higher levels in tissues. These cells do not produce virus constitutively but can be induced by activation to produce infectious virus.

There remains a need in the art for effective compositions and methods for the treatment of HIV, and particularly the eradication of persistent infection.

### SUMMARY OF THE INVENTION

In one aspect, provided herein is a lipid nanoparticle (LNP) encapsulating an mRNA comprising a nucleotide sequence encoding an HIV Tat protein. In certain embodiments, the HIV tat protein comprises the amino acid sequence of SEQ ID NO: 1, or a fragment of the sequence of SEQ ID NO: 1. In certain embodiments, the sequence encoding the HIV tat protein comprises the sequence of SEQ ID NO: 2, or a sequence sharing at least 70% identity with SEQ ID NO: 2. In certain embodiments, the lipid nanoparticle further comprises an antibody or peptide that specifically binds CD4, CCR5, or CXCR4 on a target immune cell.

In another aspect, provided herein is a composition comprising a nanoparticle and a pharmaceutically acceptable carrier. In certain embodiments, composition is formulated for intravenous delivery.

In another aspect, provided herein is a method of treating HIV in a subject in need thereof, the method comprising administering a nanoparticle or a composition comprising a nanoparticle described herein.

In another aspect, provided herein is a method of reactivating HIV in a latently infected immune cell, the method comprising administering a nanoparticle or a composition comprising a nanoparticle described herein, optionally wherein the immune cell is a CD4+ T cell. In certain embodiments, contacting the immune cell with the nanoparticle does not result in activation of the immune cell, optionally wherein activation of the immune cell is determined by measuring surface expression of one or more of CD69, CD25, HLA-DR, CD71, CD26, CD27, CD28, CD30, CD154, CD40L, CD134, CD44, and CD62L.

In yet another aspect, uses of the described nanoparticles or compositions in the manufacture of a medicament for treating HIV are provided.

Still other aspects and advantages of these compositions and methods are described further in the following detailed description of the preferred embodiments thereof.

### BRIEF DESCRIPTION OF THE DRAWINGS

FIG. 1A-FIG. 1B show Tat-encoding modified mRNAs are functional in vitro. Modified mRNAs were delivered into TZMbl cells seeded at 90,000 cells per well in 96-well flat bottom plates. The Mirius Trans-it transfection reagent was used according to the manufacture's protocol to deliver modified mRNA products at 1000 ng, 100 ng, 10 ng, or 1 ng per well. Each condition was performed in triplicate. (FIG. 1A) The number of GFP+ cells as counted by the Celigo live cell imager. (FIG. 1B) Substrate was added to lysed TZMbl cells and luciferase activity was measured as relative light units.

FIG. 2A-FIG. 2C show CD4-targeted Tat nanoparticles efficiently reactivate latent HIV from Jurkat T cell clones. Modified mRNAs were encapsulated in lipid nanoparticles, then subsequently incorporated with anti-human CD4 targeting antibodies or left unmodified. 24-hours after nanoparticle delivery, cells were stained for viability and CD4 and analyzed for GFP expression by flow cytometry. (FIG. 2A) CEM-GXR25 cells, a T cell line expressing GFP under an LTR promoter was treated with various amounts of either CD4-targeted or unmodified nanoparticles expressing Tat or ZsGreen. (FIG. 2B, FIG. 2C) JLat clones carrying a single integrated HIV-1 provirus were similarly treated with targeted and untargeted nanoparticles. GFP is a readout of HIV provirus transcription as the HIV Env gene has been replaced with the GFP coding sequence.

FIG. 3 shows CD4-targeted Tat nanoparticles efficiently reactivate latent HIV from Jurkat T cell clones. Primary human T cells were activated with anti-CD3/CD28 dynabeads and transduced with a lentivirus encoding an LTR-driven nanoluciferase cassette. Transduced T cells were expanded for 10 days then used for assays. Cells were treated with various amounts of targeted or untargeted Tat nanoparticle, and 48 hours later, lysed and analyzed for relative light unit expression using the NanoGlo substrate (Promega).

FIG. 4A-FIG. 4B show CD4-targeted Tat nanoparticles significantly reactivate latent HIV from primary human PBMCs. (FIG. 4A) Effects of control LRA romidepsin, hCD4-targeted Tat, control hCD4-targeted zsgreen, and untargeted Tat and zsgreen on p24 viral antigen production in cell pellets and culture supernatants, as measured by p24 Simoa. Results were obtained from 20 million PBMC per condition obtained from 1 donor with HIV and stable suppression on cART. (FIG. 4B) Viability of PBMC cultures relative to PBMC before LRA treatment. No tx=no treatment control.

### DETAILED DESCRIPTION

Provided herein are nanoparticles, and compositions comprising the same, wherein the nanoparticles encapsulate a messenger RNA (mRNA) encoding an HIV tat protein. Also provided are methods for use of the same to treat HIV infection, including for treatment of a subject that has been identified as having a latent HIV infection.

As used herein, “a,” “an,” or “the” can mean one or more than one. For example, “a” cell can mean a single cell or a multiplicity of cells.

As used herein, the term “about” refers to a variant of 10% from the reference integer and values therebetween. For example, “about” 40 base pairs, includes ±4 (i.e., 36-44, which includes the integers 36, 37, 38, 39, 40, 41, 42, 43, 44). For other values, particularly when reference is to a percentage (e.g., 90% identity, about 10% variance, or about 36% mismatches), the term “about” is inclusive of all values within the range including both the integer and fractions.

Various embodiments in the specification are presented using “comprising” language, which is inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

As used herein, the term “administered in combination” or “combined administration” means that two or more agents (e.g., mRNAs) are administered to a subject at the same time or within an interval such that there can be an overlap of an effect of each agent on the patient. In some embodiments, they are administered within about 60 minutes, 30 minutes, 15 minutes, 10 minutes, 5 minutes, or 1 minute of one another. In some embodiments, the administrations of the agents are spaced sufficiently closely together such that a combinatorial (e.g., a synergistic) effect is achieved. In some embodiments, the administration in combination can be concurrent (i.e., all the mRNAs are administered as part of a single formulation, or different mRNAs in different formulation are administered simultaneous), or consecutive (e.g., several mRNAs in several formulations are administered consecutively).

The term “amino acid substitution” refers to replacing an amino acid residue present in a parent sequence (e.g., a consensus sequence) with another amino acid residue. An amino acid can be substituted in a parent sequence, for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the nomenclature ‘AnY’, wherein A is the single letter code corresponding to the amino acid naturally or originally present at position n, and Y is the substituting amino acid residue. In other aspects, substitution patterns can be described according to the nomenclature An(YZ), wherein A is the single letter code corresponding to the amino acid residue substituting the amino acid naturally or originally present at position X, and Y and Z are alternative substituting amino acid residue.

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

When used with respect to two or more moieties, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Codon optimization refers to various approaches designed to improve the codon composition of a recombinant gene based on various criteria without altering the amino acid sequence. Various methods of codon optimization are known in the art.

As used herein, the term “identity” refers to the overall monomer conservation between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. When comparing DNA and RNA, thymine (T) and uracil (U) can be considered equivalent.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc. Unless otherwise specified, the percentage of identity values disclosed in the present application are obtained by using the implementation of MAFFT (Multiple Alignment using Fast Fourier Transform) version 7 available at the European Bioinformatics Institute (www.ebi.ac.uk/Tools/msa/mafft/with default parameters.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity “% ID” of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

The terms “nucleic acid sequence,” “nucleotide sequence,” or “polynucleotide sequence” are used interchangeably and refer to a contiguous nucleic acid sequence. The sequence can be either single stranded or double stranded DNA or RNA, e.g., an mRNA.

The phrase “nucleotide sequence encoding” refers to the nucleic acid (e.g., an mRNA or DNA molecule) coding sequence which encodes a polypeptide. The coding sequence can further include initiation and termination signals operably linked to regulatory elements including a promoter and polyadenylation signal capable of directing expression in the cells of an individual or mammal to which the nucleic acid is administered. The coding sequence can further include sequences that encode signal peptides.

The term “expression” is used herein in its broadest meaning and comprises the production of RNA, of protein, or of both RNA and protein. With respect to RNA, the term “expression” or “translation” relates in particular to the production of peptides or proteins. Expression may be transient or may be stable.

As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In certain embodiments, the synthetic mRNA comprises at least one unnatural nucleobase. In certain embodiments, all nucleobases of a certain class have been replaced with unnatural nucleobases (e.g., all uridines in a polynucleotide disclosed herein can be replaced with an unnatural nucleobase, e.g., 5-methoxyuridine). In certain embodiments, the polynucleotide (e.g., a synthetic RNA or a synthetic DNA) comprises only natural nucleobases, i.e., A, C, T and U in the case of a synthetic DNA, or A, C, T, and U in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding RNA sequences (comprising U) are considered codon-optimized nucleotide sequence of the present disclosure. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ‘P’P codon (RNA map in which U has been replaced with pseudouridine).

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al., 1993, J. Am. Chem. Soc. 115:4461-4467 and references cited therein; and isoguanine nucleotides can be prepared using the method described by Switzer et al., 1993, supra, and Mantsch et al., 1993, Biochem. 14:5593-5601, or by the method described in U.S. Pat. No. 5,780,610 to Collins et al. Other nonnatural base pairs can be synthesized by the method described in Piccirilli et al., 1990, Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

The terms “RNA in vitro transcription” or “in vitro transcription” relate to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which according to the present invention is preferably a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In a preferred embodiment of the present invention the DNA template is linearized with a suitable restriction enzyme, before it is transcribed in vitro. The cDNA may be obtained by reverse transcription of mRNA or chemical synthesis. Moreover, the DNA template for in vitro RNA synthesis may also be obtained by gene synthesis.

Methods for in vitro transcription are known in the art (see, e.g., Geall et al. (2013) Semin. Immunol. 25(2): 152-159; Brunelle et al. (2013) Methods Enzymol. 530:101-14). Reagents used in said method typically include:
1) a linearized DNA template with a promoter sequence that has a high binding affinity for its respective RNA polymerase such as bacteriophage-encoded RNA polymerases;
2) ribonucleoside triphosphates (NTPs) for the four bases (adenine, cytosine, guanine and uracil);
3) optionally a CAP analogue as defined above (e.g. m7G(5′)ppp(5′)G (m7G));
4) a DNA-dependent RNA polymerase capable of binding to the promoter sequence within the linearized DNA template (e.g. T7, T3 or SP6 RNA polymerase);
5) optionally a ribonuclease (RNase) inhibitor to inactivate any contaminating RNase;
6) optionally a pyrophosphatase to degrade pyrophosphate, which may inhibit transcription;
7) MgCl₂, which supplies Mg²⁺ ions as a co-factor for the polymerase;
8) a buffer to maintain a suitable pH value, which can also contain antioxidants (e.g. DTT), and/or polyamines such as spermidine at optimal concentrations.

As used herein, the term “polypeptide” refers to a polymer of amino acid residues typically joined by peptide bonds that can be produced naturally (e.g., isolated or purified) or synthetically.

As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells). As used herein, “T cell” includes all types of immune cells expressing CD3 including T-helper cells (CD4+cells), cytotoxic T cells (CD8+cells), T-regulatory cells (Treg) and gamma-delta T cells. Reference to a “CD” or cluster of differentiation marker, such as CD3+, CD4+, CD8+, CD56+ herein, relates to surface expression of such polypeptide.

“Tat” and “HIV Tat,” as used herein, refers to the viral protein Tat (trans-activator of transcription), which modulates the activity of the viral promoter. Tat recognizes a short-stem loop structure, known as the transactivation response element (TAR), located at the 5′ terminus of the viral transcript. Tat binding activates the transcription complex that assembles onto the viral promoter, leading to a strong increase in viral transcripts. In addition to promoting viral transcription Tat regulates the expression of cellular genes, modulating key pathways and mechanisms to generate an environment that favors the production and spread of HIV. Tat is a small basic protein coded by two exons whose length varies between 99 and 103 amino acids with the predominant form being 101 residues.

“Fragments” of proteins or peptides in the context of the present invention may, typically, comprise a sequence of a protein or peptide as defined herein, which is, with regard to its amino acid sequence (or its encoded nucleic acid molecule), N-terminally and/or C-terminally truncated compared to the amino acid sequence of the original (native) protein (or its encoded nucleic acid molecule). Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide.

A fragment of a protein may typically comprise an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective naturally occurring full-length protein.

Fragments of proteins or peptides may furthermore comprise a sequence of a protein or peptide as defined herein, which has a length of for example at least 5 amino acids, preferably a length of at least 6 amino acids, preferably at least 7 amino acids, more preferably at least 8 amino acids, even more preferably at least 9 amino acids; even more preferably at least 10 amino acids; even more preferably at least 11 amino acids; even more preferably at least 12 amino acids; even more preferably at least 13 amino acids; even more preferably at least 14 amino acids; even more preferably at least 15 amino acids; even more preferably at least 16 amino acids; even more preferably at least 17 amino acids; even more preferably at least 18 amino acids; even more preferably at least 19 amino acids; even more preferably at least 20 amino acids; even more preferably at least 25 amino acids; even more preferably at least 30 amino acids; even more preferably at least 35 amino acids; even more preferably at least 50 amino acids; or most preferably at least 100 amino acids. For example such fragment may have a length of about 6 to about 20 or even more amino acids, e.g. fragments as processed and presented by MHC class I molecules, preferably having a length of about 8 to about 10 amino acids, e.g. 8, 9, or 10, (or even 6, 7, 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T-cells in form of a complex consisting of the peptide fragment and an MHC molecule, i.e., the fragments are typically not recognized in their native form. Fragments of proteins or peptides may comprise at least one epitope of those proteins or peptides. Furthermore, domains of a protein, like the extracellular domain, the intracellular domain or the transmembrane domain and shortened or truncated versions of a protein may be understood to comprise a fragment of a protein.

“Variants” of proteins or peptides as defined in the context of the present invention may be generated, having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined in the context of the present invention may comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

A “variant” of a protein or peptide may have at least 70%, 75%, 80%, 85%, 90%, 95%, 98% or 99% amino acid identity over a stretch of 10, 20, 30, 50, 75 or 100 amino acids of such protein or peptide.

Furthermore, variants of proteins or peptides as defined herein, which may be encoded by a nucleic acid molecule, may also comprise those sequences, wherein nucleotides of the encoding nucleic acid sequence are exchanged according to the degeneration of the genetic code, without leading to an alteration of the respective amino acid sequence of the protein or peptide, i.e. the amino acid sequence or at least part thereof may not differ from the original sequence in one or more mutation(s) within the above meaning.

As used herein, the term ligand (sometimes referred to herein as binding moiety) refers to any molecule that specifically binds to another molecule, which is sometimes referred to herein as the partner molecule or target. In one embodiment, the binding moiety is an antibody. As used herein, an “antibody” is a monoclonal antibody, a synthetic antibody, a recombinant antibody, a chimeric antibody, a humanized antibody, a human antibody, a CDR-grafted antibody, a multispecific binding construct that can bind two or more targets, a dual specific antibody, a bi-specific antibody or a multi-specific antibody, or an affinity matured antibody, a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, an Fc construct, a monovalent or bivalent construct from which domains non-essential to monoclonal antibody function have been removed, a single-chain molecule containing one V_{L}, one V_{H} antigen-binding domain, and one or two constant “effector” domains optionally connected by linker domains, a univalent antibody lacking a hinge region, a single domain antibody, a dual variable domain immunoglobulin (DVD-Ig) binding protein or a nanobody. Also included in this definition are antibody mimetics such as affibodies, i.e., a class of engineered affinity proteins, generally small (~6.5 kDa) single domain proteins that can be isolated for high affinity and specificity to any given protein target.

The term “antibody fragment” as used herein for the described methods and compositions refers to less than an intact antibody structure having antigen-binding ability. Examples of antibody fragments include Fab, Fab′, F(ab′)₂, and Fv fragments; diabodies; linear antibodies; single-chain antibody molecules such as e.g. single chain Fab, scFv, and multispecific antibodies formed from antibody fragments. The “single chain Fab” format is described, e.g., in Hust M. et al. BMC Biotechnol. 2007 Mar. 8; 7:14. scFvV constructs include complementary scFvs produced as a single chain (tandem scFvs) or bispecific tandem scFvs.

An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations.

The term antibody also encompasses molecules comprising an immunoglobulin domain from an antibody (e.g., a VH, CL, CL, CH1, CH2 or CH3 domain) fused to other molecules, i.e., fusion proteins. In some embodiments, such fusion protein comprises an antigen-binding moiety (e.g., an scFv). The antibody moiety of a fusion protein comprising g an antigen-binding moiety can be used to direct a therapeutic agent (e.g., a cytotoxin) to a desired cellular or tissue location determined by the specificity of the antigen-binding moiety.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a nonhuman species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al, Nature 321:522-525 (1986); Riechmann et al, Nature 332:323-329 (1988); and Presta, Curr. Op. Struct. Biol 2:593-596 (1992).

As used herein, “specifically binding,” “binds specifically to,” “specific binding” refer, for example, to an antibody selectively or preferentially binding to an antigen. For example, with respect to a targeting moiety (such as an antibody), specifically binding refers to preferential binding refers to the ability of the antibody to bind one or more epitopes of an antigen or binding partner of interest without substantially recognizing and binding other molecules in a sample or environment containing a mixed population of antigens. Specific binding interactions are mediated by one or, typically, more noncovalent bonds between the binding molecules or binding partners.

“Patient” or “subject” or “individual” as used herein means a mammalian animal, including a human, a veterinary or farm animal, a domestic animal or pet, and animals normally used for clinical research. In certain embodiments, the subject of these methods and compositions is a human. In certain embodiments, the subject is an HIV-infected subject who is symptomatic or non-symptomatic. In certain embodiments, the subject is a human who has received, or is receiving, anti-retroviral therapy (ART). In another embodiment, the subject is a human who has discontinued ART.

As used herein, the term “treatment” refers to any method used to alleviate, delay onset, reduce severity or incidence, or yield prophylaxis of one or more symptoms or aspects of an HIV infection. For the purposes of the present invention, treatment can be administered before, during, and/or after the onset of symptoms. In certain embodiments, treatment occurs after the HIV+ subject has received ART. In some embodiments, the term “treating” includes abrogating, substantially inhibiting, slowing, or reversing the progression of a condition, substantially ameliorating clinical or aesthetical symptoms of a condition, or substantially preventing the appearance of clinical or aesthetical symptoms of a condition, or decreasing the severity and/or frequency one or more symptoms resulting from the disease.

The term “therapeutically effective amount” or “effective amount” refers to an amount agent that when administered alone or in combination with an additional therapeutic agent to a cell, tissue, or subject is effective to prevent or ameliorate HIV infection, or amelioration of persistent HIV infection. A therapeutically effective dose further refers to that amount of the LNP or LNP composition sufficient to result in the reduction, prevention, or inhibition of HIV trafficking and progression. In certain embodiments, therapeutically effective dose further refers to that amount of the LNP or LNP composition sufficient to reactivate latent HIV infection in a target cell(s).

By “receptor, epitope or protein expressed on HIV-infected cells” is meant one or more of the receptors, CD4, CCR5 or CXCR4, among others (see the receptors listed in Clapham P R and McKnight, A., HIV-1 Receptors and Cell Tropism, Brit. Med Bull., 58(1):43-59 (September 2001) incorporated herein by reference, including mutations of these receptors. By “CD4” is meant a glycoprotein of approximately 60,000 molecular weight that is expressed primarily on the cell membrane of mature, thymus-derived (T) lymphocytes, and to a lesser extent on monocyte/macrophage lineage cells. The CD4 glycoprotein plays a role in mediating cellular immunity and also serves as the receptor for HIV. By “CCR5 or CXCR4” are meant chemokine co-receptors also found on the CD4 T cell surface. Chemokine receptor 5 (CCR5), is used by macrophage-tropic (M-tropic) HIV to bind to a cell. About 90% of all HIV infections involve the M-tropic HIV strain. CXCR4, also called fusin, is a glycoprotein-linked chemokine receptor used by T-tropic HIV (ones that preferentially infect CD4 T-cells) to attach to the host cell.

### Compositions

Provided herein are compositions that include a lipid nanoparticle (LNP) encapsulated messenger RNA (mRNA) that includes a nucleotide sequence that encodes a Tat protein. In certain embodiments, the LNP includes a targeting moiety on its surface to improve delivery of the Tat encoding mRNA to a target cell, in particular an HIV infected T cell.

The native Tat protein sequence is provided below, and in SEQ ID NO: 1.

MEPVDPSLEPWKHPGSQPKTACTNCYCKKCCLHCQVCFTTKGLGISYG
RKKRRQRRRPPQDSQTHQVSLPKQPSSQQRGDPTGPKESKKKVERETE
TDPDN (UniProtKB/Swiss-Prot: P20879.1)

In certain embodiments, the encoded polypeptide is a fragment of the full-length Tat protein, such as that provided in SEQ ID NO: 1. Such fragments include N-terminal and C-terminal truncations of at least, or up to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, residues. In certain embodiments, the encoded polypeptide shares at least 70%, at least 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity with a sequence of SEQ ID NO: 1, either a partial sequence or the full-length sequence. In certain embodiments, the fragment is Tat protein having an N-terminal truncation of at least, or up to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 residues. In certain embodiments, the fragment is Tat protein having a C-terminal truncation of at least, or up to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 residues.

The nucleotides sequence encoding Tat is provided as a messenger RNA (mRNA).

In certain embodiments, the mRNA includes the following sequence or, or in the case of a Tat fragment, a partial sequence of the following:

(SEQ ID NO: 2)
AUGGAGCCAGUAGAUCCUAGCCUAGAGCCCUGGAAGCAUCCAGGAAGU
CAGCCUAAGACUGCUUGUACCAAUUGCUAUUGUAAAAAGUGUUGCCUU
CAUUGCCAAGUUUGUUUCACAACAAAAGGCUUAGGCAUCUCCUAUGGC
AGGAAGAAGCGGAGACAGCGACGAAGACCUCCUCAAGACAGUCAGACU
CAUCAAGUUUCUCUACCAAAGCAACCCUCCUCCCAGCAACGAGGGGAC
CCGACAGGCCCGAAGGAAUCGAAGAAGAAGGUGGAGAGAGAGACAGAG
ACAGAUCCGGACAAU

In certain embodiments the mRNA includes a sequence sharing at least 70%, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity with SEQ ID NO: 2. In certain embodiments, the mRNA encodes SEQ ID NO: 1. In certain embodiments, the mRNA encodes a fragment of SEQ ID NO: 1. In certain embodiments, the mRNA encodes a polypeptide having 1, 2, 3, 5, 6, 7, 8, 9, 10 or mismatches when aligned with SEQ ID NO: 1

An mRNA may include a 5′ untranslated region, a 3′ untranslated region, a Tat-encoding sequence and/or a polyA sequence. An mRNA may be a naturally or non-naturally occurring mRNA. An mRNA may include one or more modified nucleobases, nucleosides, or nucleotides. In some embodiments, the mRNA in the compositions of the invention comprise at least one modification which confers increased or enhanced stability to the nucleic acid, including, for example, improved resistance to nuclease digestion in vivo. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine.

As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA.

As used herein, the terms “stable” and “stability” as such terms relate to the nucleic acids of the present invention, and particularly with respect to the mRNA, refer to increased or enhanced resistance to degradation by, for example nucleases (i.e., endonucleases or exonucleases) which are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. The stabilized mRNA molecules provided herein demonstrate longer half-lives relative to their naturally occurring, unmodified counterparts (e.g. the wild-type version of the mRNA). In some embodiments, the mRNA exhibits increased stability including resistance to nucleases, thermal stability, and/or increased stabilization of secondary structure. In some embodiments, increased stability exhibited by the mRNA is measured by determining the half-life of the mRNA (e.g., in a plasma, cell, or tissue sample) and/or determining the area under the curve (AUC) of the protein expression by the mRNA over time (e.g., in vitro or in vivo). An mRNA is identified as having increased stability if the half-life and/or the AUC is greater than the half-life and/or the AUC of a corresponding wild-type mRNA under the same conditions.

Also contemplated by the terms “modification” and “modified” as such terms related to the mRNA of the present invention are alterations which improve or enhance translation of mRNA nucleic acids, including for example, the inclusion of sequences which function in the initiation of protein translation (e.g., the Kozak consensus sequence).

In some embodiments, the mRNA described herein have undergone a chemical or biological modification to render them more stable. Exemplary modifications to an mRNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring mRNA, for example, covalent modifications such as the introduction of modified nucleotides, (e.g., nucleotide analogs, or the inclusion of pendant groups which are not naturally found in such mRNA molecules).

In some embodiments, the number of C and/or U residues in an mRNA sequence is reduced. In another embodiment, the number of C and/or U residues is reduced by substitution of one codon encoding a particular amino acid for another codon encoding the same or a related amino acid. Contemplated modifications to the mRNA nucleic acids of the present invention also include the incorporation of pseudouridine (W) or 5-methylcytosine (m5C). Substitutions and modifications to the mRNA of the present invention may be performed by methods readily known to one or ordinary skill in the art.

In certain embodiments, the mRNA includes a 5′ cap structure, a chain terminating nucleotide, a stem loop, a polyA sequence, and/or a polyadenylation signal. A 5′-CAP is an entity, typically a modified nucleotide entity, which generally “caps” the 5′-end of a mature mRNA. A 5′-CAP may typically be formed by a modified nucleotide, particularly by a derivative of a guanine nucleotide. Preferably, the 5′-CAP is linked to the 5′-terminus via a 5′-5′-triphosphate linkage. A 5′-CAP may be methylated, e.g., m7GpppN, wherein N is the terminal 5′ nucleotide of the nucleic acid carrying the 5′-CAP, typically the 5′-end of an mRNA. m7GpppN is the 5′-CAP structure, which naturally occurs in mRNA transcribed by polymerase II. Accordingly, a mRNA sequence as described herein may comprise a m7GpppN as 5′-cap.

Further examples of 5′-CAP structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

Additional modified 5′-cap structures are cap1 (methylation of the ribose of the adjacent nucleotide of m7G), cap2 (additional methylation of the ribose of the 2nd nucleotide downstream of the m7G), cap3 (additional methylation of the ribose of the 3rd nucleotide downstream of the m7G), cap4 (methylation of the ribose of the 4th nucleotide downstream of the m7G), ARCA (anti-reverse CAP analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine. The mRNA may instead or additionally include a chain terminating nucleoside.

In certain embodiments, the mRNA includes a stem loop, such as a histone stem loop. A stem loop may include 1, 2, 3, 4, 5, 6, 7, 8, or more nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

In certain embodiments, the mRNA includes a polyA sequence. According to a further preferred embodiment, the mRNA compound comprising an mRNA sequence of the present invention may contain a poly-A tail on the 3′-terminus of typically about 10 to 200 adenosine nucleotides, about 10 to 100 adenosine nucleotides, about 40 to 80 adenosine nucleotides, or about 50 to 70 adenosine nucleotides.

In certain embodiments, the poly(A) sequence in the mRNA is derived from a DNA template by RNA in vitro transcription. Alternatively, the poly(A) sequence may also be obtained in vitro by common methods of chemical-synthesis without being necessarily transcribed from a DNA-progenitor. Moreover, poly(A) sequences, or poly(A) tails may be generated by enzymatic polyadenylation of the RNA according to the present invention using commercially available polyadenylation kits and corresponding protocols known in the art.

Alternatively, the mRNA as described herein optionally comprises a polyadenylation signal, which is defined herein as a signal, which conveys polyadenylation to a (transcribed) RNA by specific protein factors (e.g., cleavage and polyadenylation specificity factor (CPSF), cleavage stimulation factor (CstF), cleavage factors I and II (CF I and CF II), poly(A) polymerase (PAP)). In this context, a consensus polyadenylation signal is preferred comprising the NN(U/T)ANA consensus sequence. In a particularly preferred aspect, the polyadenylation signal comprises one of the following sequences: AA(U/T)AAA or A(U/T)(U/T)AAA (wherein uridine is usually present in RNA and thymidine is usually present in DNA).

In some embodiments, the mRNA sequence comprises at least one 5′- or 3′-UTR element. In this context, an UTR element includes a nucleic acid sequence, which is derived from the 5′- or 3′-UTR of any naturally occurring gene or which is derived from a fragment, a homolog or a variant of the 5′- or 3′-UTR of a gene. Preferably, the 5′- or 3′-UTR element used according to the present invention is heterologous to the at least one coding region of the mRNA sequence of the invention. Even if 5′- or 3′-UTR elements derived from naturally occurring genes are preferred, also synthetically engineered UTR elements may be used.

The term “3′-UTR element” typically refers to a nucleic acid sequence, which comprises or consists of a nucleic acid sequence that is derived from a 3′-UTR or from a variant of a 3′-UTR. A 3′-UTR element may represent the 3′-UTR of an RNA, preferably an mRNA. Thus, as used herein, a 3′-UTR element may be the 3′-UTR of an RNA, e.g., of an mRNA, or it may be the transcription template for a 3′-UTR of an RNA. Thus, a 3′-UTR element preferably is a nucleic acid sequence which corresponds to the 3′-UTR of an RNA, preferably to the 3′-UTR of an mRNA, such as an mRNA obtained by transcription of a genetically engineered vector construct. Preferably, the 3′-UTR element fulfils the function of a 3′-UTR or encodes a sequence which fulfils the function of a 3′-UTR.

The mRNA is encapsulated in a lipid nanoparticle (LNP). The term “lipid nanoparticle”, also referred to as LNP, refers to a particle having at least one dimension on the order of nanometers (e.g., 1-1,000 nm) which includes one or more lipids (e.g., cationic lipids, non-cationic lipids, and PEG-modified lipids). In some embodiments, such lipid nanoparticles comprise a cationic lipid and one or more excipient selected from neutral lipids, charged lipids, steroids and polymer conjugated lipids (e.g., a pegylated lipid). In some embodiments, the mRNA, or a portion thereof, is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells. In some embodiments, the mRNA or a portion thereof is associated with the lipid nanoparticles. Preferably, the lipid nanoparticles are formulated to deliver one or more mRNA to one or more target cells (i.e., immune cells, such as CD4+ T cells).

In the context of the present invention, lipid nanoparticles are not restricted to any particular morphology, and should be interpreted as to include any morphology generated when a cationic lipid and optionally one or more further lipids are combined, e.g., in an aqueous environment and/or in the presence of a nucleic acid compound. For example, a liposome, a lipid complex, a lipoplex and the like are within the scope of a lipid nanoparticle.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, the mRNA, when present in the lipid nanoparticles, is resistant in aqueous solution to degradation with a nuclease. As used herein, the mean diameter may be represented by the z-average as determined by dynamic light scattering.

An LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In one embodiment, the mRNA-comprising LNP comprises one or more ionizable cationic lipids as described herein, cholesterol, a helper phospholipid, and a polyethylene glycol-modified lipid.

As mentioned, the LNP comprises an ionizable cationic lipid. The cationic lipid is preferably ionizable, i.e., it becomes protonated as the pH is lowered below the pKa of the ionizable group of the lipid, but is progressively more neutral at higher pH values. When positively charged, the lipid is then able to associate with negatively charged nucleic acids. In certain embodiments, the cationic lipid comprises a zwitterionic lipid that assumes a positive charge on pH decrease. The LNP may comprise any lipid capable of forming a particle to which the one or more nucleic acid molecules are attached, or in which the one or more nucleic acid molecules are encapsulated.

In certain embodiments, the LNP may comprise any further cationic or ionizable lipid, i.e., any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N—(N′,N′dimethylaminoethane)-carbamoyl)cholesterol (DC-Chol), N-(1-(2,3-dioleoyloxy)propyl)N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2dimyristyloxyprop-3-yl)-N,N-dimethyl-N-hydroxyethyl ammonium bromide (DMRIE).

Other useful lipids include, without limitation, 98N12-5, C12-200, PLGA, PEG, PEG-DMG, PEGylated lipids, amino alcohol lipids, and KL22.

Additionally, a number of commercial preparations of cationic lipids are available which can be used in the present invention. These include, for example, LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the further cationic lipid is an amino lipid. Suitable amino lipids useful in the invention include those described in WO2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 4-(dimethylamino)-butanoic acid, (10Z,13Z)-1-(9Z,12Z)-9,12-octadecadien-1-yl-10,13-nonadecadien-1-yl ester (DLin-MC3-DMA), N,N-dimethyl-2,2-di-(9Z,12Z)-9,12-octadecadien-1-yl-1,3-dioxolane-4-ethanamine (DLin-KC2-DMA). See also, e.g., WO2014/089486, US 2018/0353616A1, and U.S. Pat. No. 8,853,377B2, which are incorporated by reference.

In certain embodiments, LNP formulation is performed using routine procedures comprising cholesterol, ionizable lipid, helper lipid, PEG-lipid and polymer forming a lipid bilayer around encapsulated mRNA (Kowalski et al., 2019, Mol. Ther. 27(4):710-728). In some embodiments, LNP comprises a cationic lipid (i.e. N-[1-(2,3-dioleoyloxy)propyl]-N,N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly(β-amino)esters (PBAEs). In some embodiments, the LNP comprises C14-4/DOPE/Chol/PEG-lipid. See, Rybakova Y., Kowalski P. S., Huang Y., Gonzalez J. T., Heartlein M. W., DeRosa F., et al. (2019). mRNA delivery for therapeutic anti-HER2 antibody expression in vivo. Mol. Ther. 27, 1415-1423 which is incorporated by reference. See also, e.g., WO2014/089486, US 2018/0353616A1, US2013/0037977A1, WO2015/074085A1, U.S. Pat. No. 9,670,152B2, and U.S. Pat. No. 8,853,377B2, which are incorporated by reference.

Certain LNPs useful herein include those that are described in WO 2021/077066 and WO 2021/055892, each of which is incorporated herein by reference in its entirety. Useful LNPs include those that show enhanced delivery to immune cells, in particular CD4+ T cells. LNP formulations may be varied to enhance delivery. For example, the type and ionizable lipid:mRNA ratio, molar ratio of ionizable lipid, phosopholipid, cholesterol, and PEG-lipid, etc. may be varied. In one embodiment, the LNP is one described by Kauffman, K. J.; Dorkin, J. R.; Yang, J. H.; Heartlein, M. W.; DeRosa, F.; Mir, F. F.; Fenton, O. S.; Anderson, D. G., Optimization of lipid nanoparticle formulations for mRNA delivery in vivo with fractional factorial and definitive screening designs. Nano letters 2015, 15 (11), 7300-7306, which is incorporated herein by reference. In certain embodiments, the LNPs are designed with ionizable lipid:mRNA weight ratios varying between 5:1 to 25:1. In certain embodiments, the ionizable lipid:mRNA weight ratio is 5:1, 10:1, 12.5:1, 15:1, 20:1, or 25:1.

Other LNPs have been described and are useful herein. See, e.g., WO 2016/118724, U.S. Pat. No. 10,413,618B2, U.S. Pat. No. 10,723,692B2, and U.S. Pat. No. 8,754,062B2, each of which is incorporated herein by reference.

In certain embodiments, the LNP comprises one or more additional lipids which stabilize the formation of particles during their formation. Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-lcarboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one embodiment, the neutral lipid is 1,2-distearoyl-sn-glycero-3phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In various embodiments, the LNPs further comprise a steroid or steroid analogue. In certain embodiments, the steroid or steroid analogue is cholesterol. In some of these embodiments, the molar ratio of the cationic lipid to cholesterol ranges from about 5:1 to 1:1.

The term “anionic lipid” refers to any lipid that is negatively charged at physiological pH. These lipids include phosphatidylglycerol, cardiolipin, diacylphosphatidylserine, diacylphosphatidic acid, Ndodecanoylphosphatidylethanolamines, N-succinylphosphatidylethanolamines, Nglutarylphosphatidylethanolamines, lysylphosphatidylglycerols, palmitoyloleyolphosphatidylglycerol (POPG), and other anionic modifying groups joined to neutral lipids.

In certain embodiments, the LNP comprises glycolipids (e.g., monosialoganglioside GM1).

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the LNP comprises an additional, stabilizing-lipid which is a polyethylene glycol-lipid (pegylated lipid). Suitable polyethylene glycollipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3di(tetradeca noxy)propyl)carba mate or 2,3-di(tetradecanoxy)propyl-N-(w-methoxy(polyethoxy)ethyl)carbamate. In various embodiments, the molar ratio of the cationic lipid to the pegylated lipid ranges from about 100:1 to about 25:1.

Other exemplary LNPs and their manufacture are described in the art, for example in U.S. Patent Application Publication No. U520120276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference in their entirety.

In certain embodiments, the LNP is associated with a targeting moiety that binds to a target on the surface of the HIV infected target cell, e.g., a latently infected CD4+ T cell. In certain embodiments, the ligand is an antibody or an antigen binding fragment thereof, and the target is an antigen on the surface of the target cell. In other embodiments, the target is a cell surface receptor, and the targeting moiety is its cognate ligand. Ideal targets include those preferentially expressed on immune cells including immune cells that harbor latent HIV. In certain embodiments, the target is CD4 and the targeting moiety is an antibody that specifically bind CD4. In certain embodiments, the target is CCR5 and the targeting moiety is an antibody that specifically binds CCR5. In certain embodiments, the target is CXCR4 and the targeting moiety is an antibody that specifically binds CXCR4. In certain embodiments the LNP is associated with targeting moieties that are individually specific for at least two of CD4, CCR5, and CXCR4. In certain embodiments, the targeting moieties are antibodies or antibody fragments. In certain embodiments, the targeting moieties include a peptide that specifically binds CD4, CCR5, or CXCR4 (See, e.g., WO 2007/019865—CD4 binding peptides, which is incorporated herein by reference). Anti-human CD4 antibodies are known in the art and include GK1.5, Q425, Keliximab, MAX.16H5, OKT4, RPA-T4, SK3, S3.5, L200, MT310, RPA-T4, and MEM-241, among others. Anti-human CCR5 antibodies are known in the art and include 2D7, 45531, 3A9, 2D4, and T21/8, among others. Anti-human CXCR4 antibodies are known in the art include EPUMBR3, UMB2, MAB172-SP, 12G5, PA3-305, among others.

### Pharmaceutical Compositions

In another aspect, a pharmaceutical composition is provided that contains a lipid nanoparticle (LNP) encapsulated messenger RNA (mRNA) comprising a sequence encoding an HIV Tat protein. The pharmaceutical composition contains a carrier, excipient, and/or preservative.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

In certain embodiments, the composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

Methods and agents well known in the art for making formulations are described, for example, in “Remington's Pharmaceutical Sciences,” Mack Publishing Company, Easton, Pa. Formulations may, for example, contain excipients, carriers, stabilizers, or diluents such as sterile water, saline, polyalkylene glycols such as polyethylene glycol, oils of vegetable origin, or hydrogenated napthalenes, preservatives (such as octadecyldimethylbenzyl, ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol), low molecular weight polypeptides, proteins such as serum albumin, gelatin, or immunoglobulins, hydrophilic polymers such as polyvinylpyrrolidone, amino acids such as glycine, glutamine, asparagine, histidine, arginine, and lysine, monosaccharides, disaccharides, and other carbohydrates including glucose, mannose, and dextrins, chelating agents such as EDTA, sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

These above compositions may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method.

Any suitable route of administration may be selected. Accordingly, pharmaceutical compositions may be formulated for any appropriate route of administration, for example, in the form of liquid solutions or suspensions (as, for example, for intravenous administration). Alternatively, pharmaceutical compositions may be in solid form (e.g., in the form of tablets or capsules, for example for oral administration). In some embodiments, pharmaceutical compositions may be in the form of powders, drops, aerosols, etc.

### Methods

The compositions provided herein are useful for treating HIV in a subject. In certain embodiments, the HIV is latent and administration of the LNP or LNP composition results in the reactivation of a latent HIV infection.

In certain embodiments, the compositions are administered in sufficient amounts to target infected cells and provide sufficient levels of Tat expression to provide a therapeutic benefit without undue adverse effects, or with medically acceptable physiological effects, which can be determined by those skilled in the medical arts. In certain embodiments, the administration of the nanoparticles is sufficient to target reactivation of latent HIV without significantly perturbing T cell activation state.

In certain embodiments the nanoparticle or composition described herein is administered intravenously. Optionally, routes other than intravenous administration may be used, such as, e.g., oral, intranasal, intratracheal, intraarterial, intraocular, intramuscular, subcutaneous, intradermal, and other parental routes of administration. Routes of administration may be combined, if desired.

In certain embodiments, the method further includes administration of a therapeutic agent in addition to an LNP composition. In certain embodiments, the LNP composition and additional agent are delivered essentially simultaneously via the same route of administration. In other embodiments, the LNP composition is delivered first. In other embodiments, the LNP composition is delivered subsequent to the additional agent(s).

The terms “anti-retroviral therapy” and “ART” refers to treatment of individuals infected with human immunodeficiency virus (HIV) using anti-HIV drugs. The standard treatment consists of a combination of at least three drugs (often called “highly active antiretroviral therapy” or HAART) that suppress HIV replication. Antiretroviral medicines that are often used to treat HIV include: Nucleoside/nucleotide reverse transcriptase inhibitors, also called nucleoside analogs, such as abacavir, emtricitabine, and tenofovir. These medicines are often combined for best results. Nonnucleoside reverse transcriptase inhibitors (NNRTIs), such as efavirenz, etravirine, and nevirapine. Protease inhibitors (PIs), such as atazanavir, darunavir, and ritonavir. Entry inhibitors, such as enfuvirtide and maraviroc. Integrase inhibitors, such as dolutegravir and raltegravir. In one embodiment, ART is a combination of drugs efavirenz, tenofovir, and emtricitabine. Other combinations, without limitation, include: Dolutegravir, abacavir and lamivudine, Dolutegravir, tenofovir and emtricitabine, elvitegravir, cobicistat and tenofovir, and emtricitabine, raltegravir, tenofovir and emtricitabine, or ritonavir-boosted darunavir, tenofovir and emtricitabine.

In certain embodiments, the method includes a step of administering to the subject receiving a composition that includes LNP described herein ART. This administration of the LNP composition occurs before the subject receives ART. In another embodiment, the administration of the LNP composition occurs after the subject receives ART. In still another embodiment, the administration of the LNP composition occurs during the course of ART. In one embodiment, it is anticipated that the administration of the LNP composition will occur as a single dose. In another embodiment, the administration is in multiple separate dosages. In certain embodiments, the methods involves administering the LNP composition for a set period of time; stopping the LNP composition, but continuing ART.

All scientific and technical terms used herein have their known and normal meaning to a person of skill in the fields of biology, biotechnology and molecular biology and by reference to published texts, which provide one skilled in the art with a general guide to many of the terms used in the present application. However, for clarity, certain terms are defined as provided herein.

### EXAMPLES

The following examples disclose specific embodiments of preparation of compositions of this invention, their characteristics, and methods of use thereof. These examples should be construed to encompass any and all variations that become evident as a result of the teachings provided herein.

### Example 1

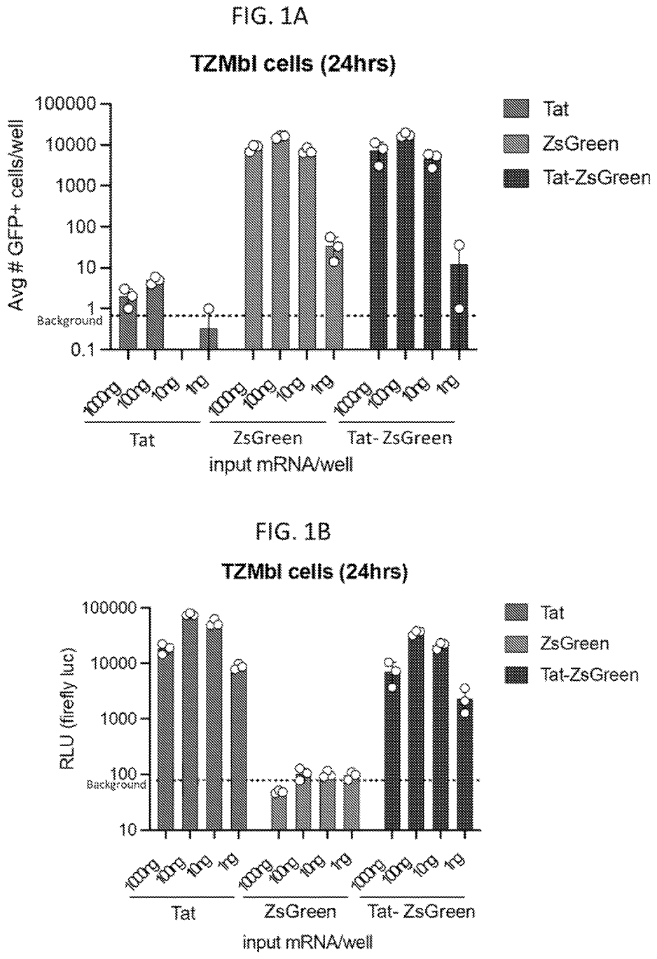

Goal: Test activity of modified mRNAs for robust expression and LTR transactivation in a HeLa-based cell line (TZMbl). The TZMbl cell line over-expresses CD4 and CCR5, and also expresses Firefly luciferase under and HIV-1 LTR (long terminal repeat) promoter. The following modified mRNAs were generated are tested:
1) A ZsGreen (GFP) encoding control
2) A Tat encoding mRNA
3) A Tat-ZsGreen fusion protein encoding mRNA
   Results: We observed efficient delivery and expression of modified mRNAs into TZMbl cells. Both the ZsGreen and Tat-ZsGreen mRNA constructs demonstrated significant ZsGreen expression over background in amounts as low as Ing (FIG. 1A). Additionally, Tat-encoding mRNA resulted in efficient transactivation of the LTR promoter as measured by relatively light units (RLU) from Firefly luciferase expression (FIG. 1B).

### Example 2

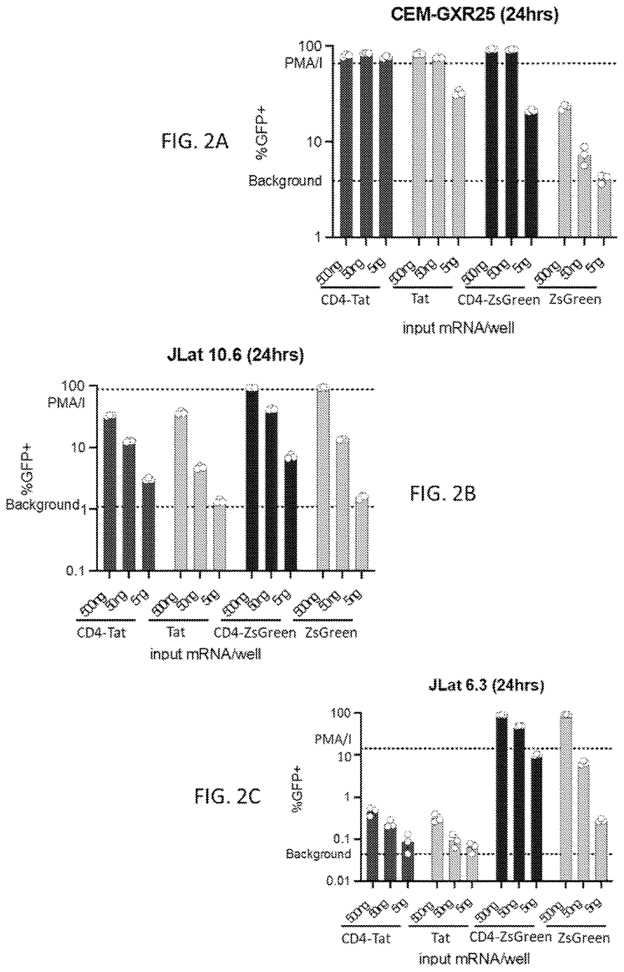

Goal: This experiment utilized a targeted nanoparticle approach, where either the Tat or ZsGreen (control) encoding mRNAs were encapsulated in unmodified or modified/CD4-targeted nanoparticles. CD4 targeting was achieved by incorporating an anti-CD4 antibody into the lipid nanoparticle. This experiment sough to test whether CD4-targeting enhanced uptake by CD4+ cells, and whether Tat expression could reactivate latently infected clonal T cell lines. The CEM-GXR25 cell line expresses CD4, CCR5, and GFP under an LTR promoter. The JLat clones contain a single integrated HIV provirus expressing GFP in the place of HIV Envelope. Four nanoparticle encapsulated mRNAs were tested:
1) unmodified ZsGreen nanoparticle
2) CD4-targeted ZsGreen nanoparticle
3) unmodified Tat nanoparticle
4) CD4-targeted Tat nanoparticle
   Results: CD4-targeted nanoparticles more efficiently entered a CD4+ T cell line (CEM-GXR25) than unmodified nanoparticles, as evidenced by greater GFP expression at the lower 5 ng mRNA input condition (FIG. 2A). JLat clone 10.6 (FIG. 2B) and 6.3 (FIG. 2C) represent easier or more difficult to reactivate T cell clones, respectively. Tat encoding nanoparticles drove significant GFP expression over background, demonstrating the ability to reactivate latent HIV-1 proviruses in vitro (FIG. 2B and FIG. 2C).

### Example 3

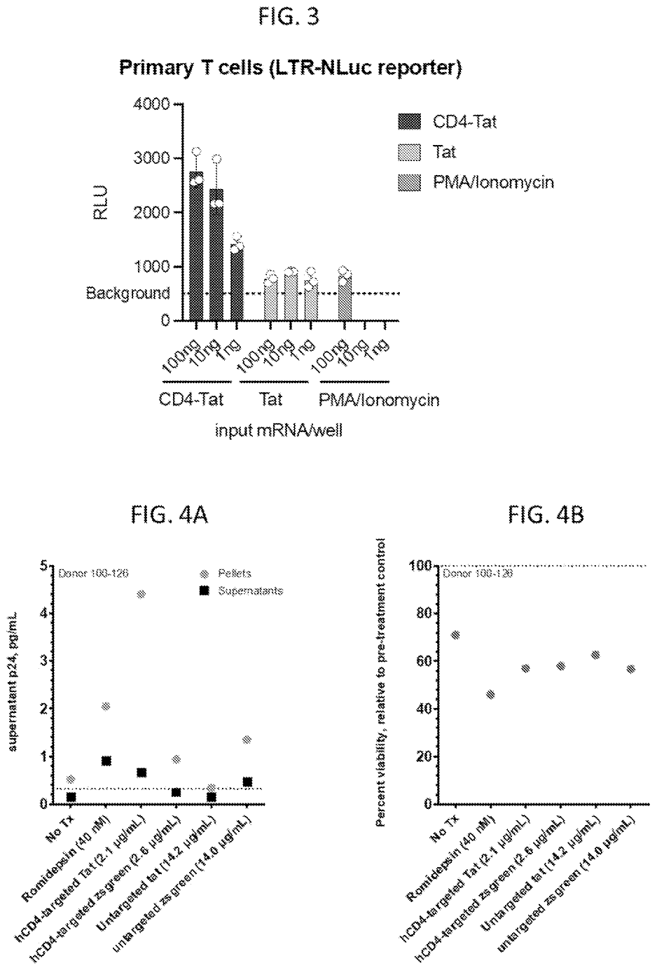

Goal: This experiment utilized targeted LNPs to test whether CD4-targeted nanoparticles could efficiently transfect primary CD4+ T cells engineered to express an LTR-driven nanoluciferase reporter.
Results: CD4-targeted nanoparticle led to significant LTR transactivation and nanoluciferase expression over background (FIG. 3). There was a dose response, and this response was greater than treatment with unmodified Tat nanoparticle or stimulation with PMA/Ionomycin.

### Example 4

Goal: Test the ability of CD4-targeted LNPs encoding Tat to reactivate replication competent proviruses from PBMCs derived from HIV-infected, ART-suppressed individuals, using a highly sensitive p24 assay.
Results: CD4-Targeted Tat mRNA nanoparticles elicited p24 production from latently infected PBMCs to a greater extent than Romidepsin (FIG. 4A), while maintaining high cell viability (FIG. 4B).

Each and every patent, patent application, and publication, including websites and other publications cited throughout the specification, is incorporated herein by reference. U.S. Provisional Patent Application No. 63/487,006, filed Feb. 26, 2023, is incorporated herein by reference. While the invention has been described with reference to particular embodiments, it will be appreciated that modifications can be made without departing from the spirit of the invention. Such modifications are intended to fall within the scope of the appended claims.

1. A lipid nanoparticle (LNP) encapsulating an mRNA comprising a nucleotide sequence encoding an HIV Tat protein.

2. The lipid nanoparticle of claim 1, wherein the HIV tat protein comprises the amino acid sequence of SEQ ID NO: 1, or a fragment of the sequence of SEQ ID NO: 1.

3. The lipid nanoparticle of claim 1, wherein the sequence encoding the HIV tat protein comprises the sequence of SEQ ID NO: 2, or a sequence sharing at least 70% identity with SEQ ID NO: 2.

4. The lipid nanoparticle of claim 1, further comprising an antibody or peptide that specifically binds CD4, CCR5, or CXCR4 on a target immune cell.

5. The lipid nanoparticle of claim 1, wherein the mRNA comprises (i) a 3′ UTR; (ii) a 5′ UTR; and/or (iii) a polyA tail.

6. The lipid nanoparticle of claim 1, wherein the polynucleotide comprises a 5′ terminal cap structure.

7. The lipid nanoparticle of claim 1, wherein the mRNA comprises at least one chemically modified nucleotide or nucleoside.

8. The lipid nanoparticle of claim 7, wherein the at least one chemically modified nucleotide or nucleoside is pseudouridine, Nl-methylpseudouridine, 5-methylcytosine, 5-methoxyuridine, or a combination thereof.

9. A composition comprising the lipid nanoparticle of claim 1 and a pharmaceutically acceptable carrier.

10. The composition of claim 9, wherein the composition is formulated for intravenous delivery.

11. A method of treating HIV in a subject in need thereof, the method comprising administering the nanoparticle of claim 1 or the composition of claim 9 to the subject.

12. The method of claim 11, further comprising administering an anti-retroviral therapy to the subject.

13. A method of reactivating HIV in a latently infected immune cell, the method comprising contacting the immune cell with the nanoparticle of claim 1 or the composition of claim 9, optionally wherein the immune cell is a CD4+ T cell.

14. The method of according to claim 13, wherein contacting the immune cell with the nanoparticle does not result in activation of the immune cell, optionally wherein activation of the immune cell is determined by measuring surface expression of one or more of CD69, CD25, HLA-DR, CD71, CD26, CD27, CD28, CD30, CD154, CD40L, CD134, CD44, and CD62L.

15-16. (canceled)


SEQUENCE LISTING (2 sequences)

SEQ ID NO: 1 - protein, 101 aa, Human immunodeficiency virus 1
MEPVDPSLEPWKHPGSQPKTACTNCYCKKCCLHCQVCFTTKGLGISYGRKKRRQRRRPPQDSQTHQVSLPKQPSSQQRGDPTGPKESKKKVERETETDPDN

SEQ ID NO: 2 - DNA, 303 bp, synthetic construct
  source 1..303: mol_type = other DNA
atggagccagtagatcctagcctagagccctggaagcatccaggaagtcagcctaagactgcttgtaccaattgctattgtaaaaagtgttgccttcattgccaagtttgtttcacaacaaaaggcttaggcatctcctatggcaggaagaagcggagacagcgacgaagacctcctcaagacagtcagactcatcaagtttctctaccaaagcaaccctcctcccagcaacgaggggacccgacaggcccgaaggaatcgaagaagaaggtggagagagagacagagacagatccggacaat